(19) United States
(12) Patent Application Publication
(10) Pub. No.: US 2026/0298851 A1
(43) Pub. Date: Oct. 1, 2026

(54) NMR MEASUREMENT OF GLYCOPROTEINS

(71) Applicant: Universität Zu Lüebeck, Luebeck (DE)

(72) Inventors: Ulrich Guenther, Badendorf (DE); Alvaro Mallagaray De Benito, Luebeck (DE); Lorena Rudolph, Luebeck (DE); Melissa Lindloge, Luebeck (DE)

(73) Assignee: Universitaet Zu Luebeck, Luebeck (DE)

(21) Appl. No.: 19/476,708

(22) PCT Filed: Apr. 19, 2024
(86) PCT No.: PCT/DE2024/100346

§ 371 Date: Oct. 19, 2025

(30) Foreign Application Priority Data
Apr. 21, 2023 (DE) 10 2023 110 285.0

(51) Int. Cl. G01N 24/08; G01N 33/68; G01R 33/46; G01R 33/465; G16B 15/00; G16H 10/40; G16H 10/60; G16H 50/20

(52) U.S. Cl. CPC G01N 24/087; G01N 33/6842; G01R 33/4625; G01R 33/465; G16B 15/00; G16H 10/40; G16H 10/60; G16H 50/20; G01N 2800/60

(57) ABSTRACT

A method for measuring the contributions of individual glycoproteins and/or glycosylation patterns as biomarkers, derived from an analysis of NMR spectra of blood serum and/or blood plasma. as well as a computer program product for subjecting biological in vitro blood plasma or serum samples to computer analysis and a method for diagnosis of a disease, comprising measuring the contributions of at least one of individual glycoproteins and glycosylation patterns as biomarkers or subjecting biological in vitro blood plasma or serum samples to computer analysis.

The present invention relates to the NMR analysis of protein signals in blood serum or plasma samples.

Nuclear magnetic resonance spectroscopy is increasingly being used to determine biomarkers, in particular lipoprotein biomarkers associated with cholesterol. See U.S. Pat. Nos. 4,933,844 A, 6,617,167 A1, and 9,470,771 B2, as well as EP 2 859 339 B1 and EP 3 588 120 B1, the contents of which are hereby incorporated by reference in their entirety. In general, to evaluate lipoproteins in blood plasma and/or serum, the intensity of various methyl and methylene signals in NMR spectra of blood and/or serum is deconvoluted to identify subclasses of lipoproteins.

Gruppen EG et al. propose GlycA as a new NMR spectroscopic biomarker for the detection of inflammation and cardiovascular disease [Gruppen EG, Riphagen IJ, Connelly MA, Otvos JD, Bakker SJL, Dullaart RPF (2015) GlycA, a Pro-Inflammatory Glycoprotein Biomarker, and Incident Cardiovascular Disease: Relationship with C-Reactive Protein and Renal Function. PLOS ONE 10 (9)].

NMR methods that use signals from glycoproteins (GlycA and GlycB) to derive medical markers for medical risk assessment are known from EP 2 859 339 B1.

The signals from GlycA and GlycB originate from a series of proteins often referred to as acute phase proteins, wherein they are characterized by their strong response to inflammation, leading to a significant increase or decrease in the blood. Acute phase proteins include CRP, serum amyloid A, haptoglobin, fibrinogen, α-1-acid glycoprotein, haptoglobin, serotransferrin, α-1-antitrypsin, and α-1-antichymotrypsin, among others. It is known that the concentration of acute phase proteins changes in a disease-specific manner not only in response to inflammatory events, but also in terms of their glycosylation pattern. The GlycA and GlycB signals in NMR spectra are based on methyl groups of specific glycan residues that are part of these acute phase proteins and which represent excellent biomarkers.

The GlycA and GlycB NMR signals have been reported to be associated with clinical-pathological conditions, such as an increased risk of mortality [Ritchie SC, Kettunen J, Brozynska M, et al. Elevated serum alpha-1 antitrypsin is a major component of GlycA-associated risk for future morbidity and mortality. Feng Y-M, ed. PLOS ONE and Kettunen J, Ritchie SC, Anufrieva O, et al. Biomarker Glycoprotein Acetyls Is Associated with the Risk of a Wide Spectrum of Incident Diseases and Stratifies Mortality Risk in Angiography Patients. Circ: Genomic and Precision Medicine. 2018;11:e002234 and Gruppen EG, Connelly MA, Sluiter WJ, Bakker SJL, Dullaart RPF. Higher plasma GlycA, a novel pro-inflammatory glycoprotein biomarker, is associated with reduced life expectancy: The PREVEND study. Clinica Chimica Acta. 2019;488:7-12.], Adiposity [Levine JA, Han JM, Wolska A, et al. Associations of GlycA and high-sensitivity C-reactive protein with measures of lipolysis in adults with obesity. Journal of Clinical Lipidology. 2020;14:667-674, Jago R, Drews KL, Otvos JD, Willi SM, Buse JB, for the HEALTHY Study Group. Novel measures of inflammation and insulin resistance are related to obesity and fitness in a diverse sample of 11-14 year olds: The HEALTHY Study. Int J Obes. 2016;40:1157-1163, Manmadhan A, Lin B-X, Zhong J, et al. Elevated GlycA in severe obesity is normalized by bariatric surgery. Diabetes Obes Metab. 2019;21:178-182], Diabetes [Connelly MA, Otvos JD, Zhang Q, et al. Effects of hepato-preferential basal insulin peglispro on nuclear magnetic resonance biomarkers lipoprotein insulin resistance index and GlycA in patients with diabetes. Biomarkers in Medicine. 2017;11:991-1001, Dullaart RPF, Gruppen EG, Connelly MA, Otvos JD, Lefrandt JD. GlycA, a biomarker of inflammatory glycoproteins, is more closely related to the leptin/adiponectin ratio than to glucose tolerance status, Akinkuolie AO, Pradhan AD, Buring JE, Ridker PM, Mora S. Novel Protein Glycan Side-Chain Biomarker and Risk of Incident Type 2 Diabetes Mellitus. ATVB. 2015;35:1544-1550], diabetes-related metabolic syndromes [Gruppen EG, Riphagen IJ, Connelly MA, Otvos JD, Bakker SJL, Dullaart RPF. GlycA, a Pro-Inflammatory Glycoprotein Biomarker, and Incident Cardiovascular Disease: Relationship with C-Reactive Protein and Renal Function. Shimosawa T, ed. PLOS ONE. 2015;10:e0139057], heart failure [Jang S, Ogunmoroti O, Ndumele CE, et al. Association of the Novel Inflammatory Marker GlycA and Incident Heart Failure and Its Subtypes of Preserved and Reduced Ejection Fraction: The Multi-Ethnic Study of Atherosclerosis. Circ: Heart Failure. 2020;13:e007067], rheumatoid arthritis [Rodriguez-Carrio J, Alperi-López M, López P, et al. GlycA Levels during the Earliest Stages of Rheumatoid Arthritis: Potential Use as a Biomarker of Subclinical Cardiovascular Disease. JCM. 2020;9:2472, Ormseth MJ, Chung CP, Oeser AM, et al. Utility of a novel inflammatory marker, GlycA, for assessment of rheumatoid arthritis disease activity and coronary atherosclerosis. Arthritis Res Ther. 2015;17:117], chronic obstructive pulmonary disease (COPD) [Prokić I, Lahousse L, de Vries M, et al. A cross-omics integrative study of metabolic signatures of chronic obstructive pulmonary disease. BMC Pulm Med. 2020;20:193], systemic lupus erythematosus [Dierckx T, Chiche L, Daniel L, Lauwerys B, Van Weyenbergh J, Jourde-Chiche N. Serum GlycA Level Is Elevated in Active Systemic Lupus Erythematosus and Correlates to Disease Activity and Lupus Nephritis Severity. JCM. 2020;9:970, Chung CP, Ormseth MJ, Connelly MA, et al. GlycA, a novel marker of inflammation, is elevated in systemic lupus erythematosus. Lupus. 2016;25:296-300], psoriasis [Joshi AA, Lerman JB, Aberra TM, et al. GlycA Is a Novel Biomarker of Inflammation and Subclinical Cardiovascular Disease in Psoriasis. Circ Res. 2016;119:1242-1253], cystic fibrosis [Kevat AC, Carzino R, Vidmar S, Ranganathan S. Glycoprotein A as a biomarker of pulmonary infection and inflammation in children with cystic fibrosis. Pediatr Pulmonol. 2020;55:401-406], polycystic ovary syndrome [Fuertes-Martin R, Moncayo S, Insenser M, et al. Glycoprotein A and B Height-to-Width Ratios as Obesity-Independent Novel Biomarkers of Low-Grade Chronic Inflammation in Women with Polycystic Ovary Syndrome (PCOS). J Proteome Res. 2019;18:4038-4045], Cushing's syndrome [Makri A, Cheung A, Sinaii N, et al. Lipoprotein particles in patients with pediatric Cushing disease and possible cardiovascular risks. Pediatr Res. 2019;86:375-381], Respiratory tract infections [Ritchie SC, Würtz P, Nath AP, et al. The Biomarker GlycA Is Associated with Chronic Inflammation and Predicts Long-Term Risk of Severe Infection. Cell Systems. 2015;1:293-301], COVID-19 [Lodge S, Nitschke P, Kimhofer T, et al. Diffusion and Relaxation Edited Proton NMR Spectroscopy of Plasma Reveals a High-Fidelity Supramolecular Biomarker Signature of SARS-COV-2 Infection. Anal Chem. 2021;93:3976-3986, Nitschke P, Lodge S, Kimhofer T, et al. J-Edited Diffusional (JEDI) Proton Nuclear Magnetic Resonance Spectroscopic Measurement of Glycoprotein and Supramolecular Phospholipid Biomarkers of Inflammation in Human Serum. :24] HIV [Tibuakuu M, Fashanu OE, Zhao D, et al. GlycA, a novel inflammatory marker, is associated with subclinical coronary disease. AIDS. 2019;33:547-557, Malo A-I, Rull A, Girona J, et al. Glycoprotein Profile Assessed by (¹⁾ H-NMR as a Global Inflammation Marker in Patients with HIV Infection. A Prospective Study. JCM. 2020;9:1344], primary aldosteronism [Berends AMA, Buitenwerf E, Gruppen EG, et al. Primary aldosteronism is associated with decreased low-density and high-density lipoprotein particle concentrations and increased GlycA, a pro-inflammatory glycoprotein biomarker. Clin Endocrinol. 2019;90:79-87], inflammatory bowel disease [Dierckx T, Verstockt B, Vermeire S, van Weyenbergh J. GlycA, a Nuclear Magnetic Resonance Spectroscopy Measure for Protein Glycosylation, is a Viable Biomarker for Disease Activity in IBD. Journal of Crohn's and Colitis. 2019;13:389-394,31], chronic kidney disease [Titan SM, Pecoits-Filho R, Barreto SM, Lopes AA, Bensenor IJ, Lotufo PA. GlycA, a marker of protein glycosylation, is related to albuminuria and estimated glomerular filtration rate: the ELSA-Brasil study. BMC Nephrol. 2017;18:367], Kawasaki disease [Connelly MA, Shimizu C, Winegar DA, et al. Differences in GlycA and lipoprotein particle parameters may help distinguish acute Kawasaki disease from other febrile illnesses in children. BMC Pediatr. 2016;16:151], cancer [Gruppen EG, Connelly MA, Dullaart RPF. Higher circulating GlycA, a pro-inflammatory glycoprotein biomarker, relates to lipoprotein-associated phospholipase A2 mass in nondiabetic subjects but not in diabetic or metabolic syndrome subjects. Journal of Clinical Lipidology. 2016;10:512-518, Gao H, Dong B, Liu X, Xuan H, Huang Y, Lin D. Metabonomic profiling of renal cell carcinoma: High-resolution proton nuclear magnetic resonance spectroscopy of human serum with multivariate data analysis. Analytica Chimica Acta. 2008;624:269-277] and decline in cognitive function [Cohen-Manheim I, Doniger GM, Sinnreich R, et al. Increase in the Inflammatory Marker GlycA over 13 Years in Young Adults Is Associated with Poorer Cognitive Function in Midlife. Wylie GR, ed. PLOS ONE. 2015;10:e0138036]. Thanks to these reports, the GlycA and GlycB signals are considered promising clinical biomarkers for clinical diseases. However, for a differentiated medical diagnosis, it would be very important to determine the individual proteins contributing to GlycA and GlycB. A detailed differentiation of the proteins would provide additional parameters for the differential diagnosis of diseases.

However, the results derived from GlycA and GlycB are sometimes nonspecific in terms of the nature of the proteins and the type of glycans. A more detailed analysis of these and other protein signals would be necessary to determine the contribution of specific acute phase proteins or at least groups of proteins.

WO 2022/157 729 A1 and EP 3 588 120 B1 uses a combination of GlycA (2.0 ppm to 2.09 ppm), GlycB (2.09 to 2.2 ppm), and a supramolecular phospholipid cluster (SPC) (3.20 to 3.30 ppm associated with (^{+)N}(CH(₃₎₎(₃₎from acetylcholine and its derivatives) as markers for identifying medical signatures to assess the medical risk associated with SARS-COV-2 infections, acute inflammation, or cardiovascular risk diseases. The determination of the percentage of individual glycoproteins or specific glycan structures and the associated direct assignment to specific diseases is not intended.

Therefore, the objective of the invention is to provide a method for measuring the contributions of individual glycoproteins and/or glycosylation patterns as biomarkers, derived from an analysis of NMR spectra of blood serum and/or blood plasma.

The task of the invention is solved by a method for measuring the contributions of individual glycoproteins and/or glycosylation patterns as biomarkers, derived from an analysis of NMR spectra of blood serum and/or blood plasma, comprising the steps:
i. selecting glycoproteins in NMR spectra from a sample of blood serum and/or blood plasma based on their specific diffusion and/or relaxation properties and/or their specific frequencies and/or coupling constants (J-couplings) in the NMR spectrum.
ii. Determination of the glycan, GlycA, and GlycB signal groups in an NMR spectrum, wherein the GlycA signal group is formed by methyl groups of Neu5Ac (5-acetyl-neuraminic acid) and wherein the GlycB signal group is formed by methyl groups of GlcNAc units and wherein the glycan signal group is between 3.5 and 5.5 ppm and the Neu5Ac-H3eq signal group is between 2.6 and 2.9 ppm and the Neu5Ac-H3ax signal group is between 1.6 and 1.88 ppm, the GlycA signal group is between 2.0 and 2.07 ppm, and the GlycB signal group is between 2.07 and 2.2 ppm, and wherein the signals are assigned on the basis of the diagram shown in FIG. 1.
iii. Adjustment of characteristic frequency matrices for individual glycoproteins or glycoproteins previously grouped into classes to the determined signal groups using mathematical methods.
iv. Determination of the contributions of individual glycoprotein biomarkers by deconvolution of the spectrum using the frequency matrices adapted in step iii and addition of a previously assigned number of mathematical functions.

In a preferred embodiment of the invention, the selection of glycoproteins from a sample of blood serum and/or blood plasma based on their specific diffusion and/or relaxation properties (step i) by means of diffusion difference spectroscopy (DDS), wherein differences in diffusion spectra with different diffusion parameters are used to select proteins of different sizes and diffusion properties.

In a further preferred embodiment of the invention, the contributions of individual glycoprotein biomarkers are determined using differences in the contributions of individual frequency matrix components.

In a further preferred embodiment, multiplication is performed with a scaling factor determined by a reference measurement to indicate concentrations in mg/ml, wherein the multiplication may be performed before the deconvolution of the spectra or after step iv.

In another aspect, the invention is embodied by a computer program product for evaluating biological in vitro blood plasma or serum samples, comprising a non-volatile computer-readable storage medium containing computer-readable program code embodied in the storage medium, wherein the computer-readable program code comprises: computer-readable program code that unfolds an NMR spectrum of a matching region of a blood plasma or serum sample from an individual, wherein the computer-readable program code unfolds the composite NMR spectrum using a characteristic frequency matrix that includes unfolding models for different proteins into signals with components of (a) GlycA, (b) GlycB, (c) glycan protons, and (d) methyl group protons, and wherein the program code defines the frequency ranges for determining the glycan, GlycA, and GlycB signal groups, wherein the glycan signal group is between 3.5 and 5.5 ppm and the Neu5Ac-H3eq signal group is between 2.6 and 2.9 ppm and the Neu5Ac-H3ax signal group between 1.6 and 1.88 ppm, the GlycA signal group between 2.0 and 2.07 ppm, and the GlycB signal group between 2.07 and 2.2 ppm, and a scheme for assigning the signals according to FIG. 1.

and wherein the program code adds a defined number of mathematical functions at frequencies known from the characteristic frequency matrix in order to determine the percentages of the individual proteins or protein groups.

The selection of glycoproteins from a blood serum sample based on their specific diffusion and/or relaxation properties (step i) is performed by editing the NMR spectrum. According to the invention, J-, T(₂₎relaxation and diffusion-edited spectra or their difference are used to edit the NMR spectrum, which is referred to here as diffusion difference spectroscopy (DDS).

Editing using the T₂relaxation method can be learned by those skilled in the art from [Carr HY, Purcell EM. Effects of Diffusion on Free Precession in Nuclear Magnetic Resonance Experiments. Phys Rev. 1954;94:630-638 and Meiboom S, Gill D. Modified Spin-Echo Method for Measuring Nuclear Relaxation Times. Review of Scientific Instruments. 1958;29:688-691].

It can also be used the method of J-Edited Diffusional (JEDI) proton NMR spectroscopy known form J-Edited Diffusional (JEDI) proton nuclear magnetic resonance spectroscopic measurement of glycoprotein and supramolecular phospholipid biomarkers of inflammation in human serum method described by [Nitschke P, Lodge S, Kimhofer T, et al. :24].

It is also possible to edit the NMR spectra according to the DIRE approach, which is based on the simplification of ¹H-NMR spectra by using differences in molecular diffusion coefficients alone and combinations of relaxation and diffusion parameters, and which is known to those skilled in the art from [Liu M, Nicholson JK, Lindon JC. High-Resolution Diffusion and Relaxation Edited One- and Two-Dimensional (^{1) H}NMR Spectroscopy of Biological Fluids. Anal Chem. 1996;68:3370-3376].

Diffusion difference spectroscopy (DDS) is defined here as the use of an NMR pulse sequence that includes an element of diffusion editing, referred to by experts as DOSY (Diffusion Editing Spectroscopy) [Liu M, Nicholson JK, Lindon JC. High-Resolution Diffusion and Relaxation Edited One- and Two-Dimensional HNMR Spectroscopy of Biological Fluids. Anal Chem. 1996; 68:3370-3376], wherein the spectra are recorded with different diffusion parameters so that the difference between individual spectra leads to the selection of a range of diffusion rates, with proteins of different diffusion parameters and, as a consequence, different sizes being imaged in each range. For DDS, two or more such spectra with different diffusion parameters can be used.

DDS enables the editing of NMR spectra for the selection of proteins with different diffusion properties, which result, for example, from the protein size. DDS can also be used to suppress the signals of small molecules (<1,000 Da) and lipoproteins in NMR spectra.

Alternatively, NMR methods can also be used in step i, which select certain frequencies and transfer them from the initial selection to other frequency ranges using a method known to experts as TOCSY (Total Correlation Spectroscopy). In this way, glycan and/or GlycA and/or GlycB signals can be selected [TOCSY: Chapter 6 in J. Cavanagh, W. J. Fairbrother, A. G. Palmer III, M. Rance and N. J. Skelton, Protein NMR Spectroscopy (2nd edition, Academic Press, 2007)].

The field of application for the method presented here is particularly medicine, especially for the diagnosis of diseases. Such diseases include, in particular, cardiogenic shock, COVID-19, obesity, diabetes, metabolic syndrome, heart failure, rheumatoid arthritis, chronic obstructive pulmonary disease (COPD), systemic lupus erythematosus, psoriasis, cystic fibrosis, polycystic ovary syndrome, Cushing's disease, respiratory tract infection, HIV, primary aldosteronism, inflammatory bowel disease, chronic kidney disease, Kawasaki disease, cognitive decline, or cancer.

FIG. 1 shows an edited ¹H-NMR spectrum of glycolysed acute phase proteins in blood serum. The glycan signal group is at 3.5 and 5.5 ppm, the Neu5Ac-H3eq signal group between 2.6 and 2.9 ppm, the Neu5Ac-H3ax signal group between 1.6 and 1.88 ppm, the fucose methyl signal group between 1.1 and 1.3 ppm, the GlycA signal group between 2.0 and 2.07 ppm, and the GlycB signal group between 2.07 and 2.2 ppm. The signals can be assigned based on the diagram shown, wherein the GlycA signal group is formed by methyl groups of Neu5Ac and wherein the GlycB signal group is formed by methyl groups of GlcNAc units.

FIG. 2 shows the typical structure of a glycan unit consisting of several sugar units, as found on the surface of glycoproteins. It is a schematic and exemplary representation of glycans that form the N-glycosylation of acute phase proteins. The glycan structures can consist of different individual glycan units, and the composition of the entire glycan arrangements can also vary. The observed methyl signals that contribute to the GlycA and GlycB regions of the spectra are marked with a square.

FIG. 3 shows a schematic representation of the individual glycan units and groups that generate NMR signals. The squares show the methyl groups that contribute to the GlycA and GlycB signals in the NMR spectra. Dotted circles show protons (H-e atoms) that contribute to the glycan signals, Neu5Ac-H3ax, Neu5Ac-H3eq, and fucose methyl in the NMR spectra.

FIG. 4 shows the composition of the GlycA and GlycB signal envelope with contributions from glycan structures found as part of protein glycosylations, specifically from the methyl groups (CH₃) of GlcNAc and Neu5Ac (marked with a square), to illustrate the allocation scheme. The GlycA signal consists mainly of Neu5Ac CH₃, while the GlycB signal arises mainly from GlcNAc; protein methionine signals also contribute to the overall signal envelope. In the structural representations of Neu5Ac, the GlcNAc and methionine methyl groups (CH₃) are wherein a square is used.

FIG. 5 shows HSQC, HMBC, and NOESY NMR spectra demonstrating the assignment of GlycA resonances to Neu5Ac methyl groups and GlycB resonances to GlcNAc methyl groups, confirming the assignment of Neu5Ac CH₃ to GlycA and GlcNAc CH₃ to GlycB. (a) NOESY spectra show that GlycA produces only cross signals with Neu5Ac (X,Z) glycan protons, while GlycB shows cross signals with protons from GlcNAc (O) corresponding to the same glycan unit and also with nearby mannose (E) and (F) protons. In (b), excerpts from a NOESY spectrum are shown, demonstrating that after removal of all Neu5Ac with neuraminidase, only cross peaks between GlycB and GlcNAc (O) and mannose (E) and (F) remain, further supporting the assignment of GlycA to Neu5Ac and GlycB to GlcNAc. (c) shows HSQC and HMBC spectra showing connections between GlycA and the associated H5 group of Neu5Ac (X/Z5, solid line) and between GlycB and the H2 group of GlcNAc (O2, dotted line). The spectral representations confirm the correctness of the assignment of the glycan units shown in FIG. 4. (d) shows a sketch summarizing the significance of the NOESY cross signals observed in (a) and (b).

FIG. 6 shows the contributions of common N-glycans, acute phase proteins in human blood, to the GlycA/B signal. (Top) GlycA/B envelope curve showing which signals are generated by methyl groups of GlcNAc (gray), Neu5Ac (black), and methionine (gray dashed line). (Bottom) Spectral range corresponding to the N-acetyl methyl groups of the most common N-glycans normally found in human plasma. Z/X denotes Neu5Ac, O/P denotes GlcNAc located on glycan branches, and J indicates the distal GlcNAc of the N-glycan group. GlcNAc η is not usually visible in NMR spectra of plasma samples. Its contribution is indicated by a thin gray dashed line in the NMR spectra. The specific glycan units that generate the NMR signals are indicated in the glycan sketches on the right. It can be seen that GlycA corresponds exclusively to Neu5Ac signals, while GlycB corresponds only with signals generated by GlcNAc.

FIG. 7 shows the contributions of low-abundance N-glycans to the GlycA/B signal. (Top) GlycA/B envelope curve showing which signals are generated by methyl groups of GlcNAc (gray), Neu5Ac (black), and methionine (gray dashed line). (Bottom) Spectral range corresponding to the N-acetyl methyl groups of the most common N-glycans normally found in human plasma. O/P denotes GlcNAc located on glycan branches, and J denotes the distal GlcNAc located on the N-glycan core. The GlcNAc η is not usually visible in NMR studies of plasma samples. This is indicated by a thin gray dashed line in the NMR spectra. The specific glycan units that generate the NMR signals are indicated in the glycan sketches on the right. It can be seen that GlycA corresponds exclusively to Neu5Ac signals, while GlycB corresponds only to signals generated by GlcNAc.

The following section explains step iii of the invention, the adaptation of characteristic frequency matrices for individual glycoproteins or glycoproteins previously grouped into classes to the specific signal groups using mathematical methods, in more detail without limiting the generality of the teaching.

The evaluation of the pulse sequences for the analysis of glycoproteins follows [Carr HY, Purcell EM. Effects of Diffusion on Free Precession in Nuclear Magnetic Resonance Experiments. Phys Rev. 1954;94:630-638].

FIG. 8 shows typical pulse sequences for the NMR analysis of glycoproteins from blood serum or plasma (a) pulse sequence with a diffusion and relaxation block using a perfect CPMG element (b) pulse sequence with a diffusion, relaxation, and J-editing block using a regular CPMG element. Pulse sequences (a) and (b) can be used with one or more echo times (N) and one or more gradient strengths (G1) for diffusion difference spectroscopy (DDS) to select signals from specific glycoprotein glycans based on their T₂- relaxation rates and diffusion regimes in difference spectra. (c) Single pulsed field gradient selective (SPFGSE) TOCSY pulse sequence (or simply sTOCSY) and (d) perfect double pulsed field gradient selective (Perfect-DPFGSE) TOCSY pulse sequence (or simply sPerTOCSY) for selecting signals for glycoprotein analysis. (e) Selective CPMG (sCPMG) and (f) selective perfect CPMG (sPerCPMG) serve the same purpose. The T₂- relaxation filter is used for selective suppression of unwanted protein signals. Both pulse sequences use a 180° amplitude- and phase-modulated waveform during the SPFGSE block so that multiple glycan signals can be co-selected.

FIG. 9 shows a typical unfolding of GlycA/B signals from an edited ¹H-NMR spectrum using line shape fitting. Intensities, areas, and positions of the fitted line shapes are used in combination with the frequency matrix and an external reference with known concentrations to quantify individual or groups of glycoproteins in the acute phase.

FIG. 10 shows an example of the fitting with a characteristic frequency matrix according to the invention for the spectral range of GlycA/B using serotransferrin as an example. A representation of the frequency matrix for serotransferrin (determined by isolating the protein from blood serum) can be seen. (Top) Fitting of the corresponding NMR spectrum after supplementing the blood serum with the isolated serotransferrin. (Bottom) Fitting of the corresponding NMR spectrum of isolated serotransferrin.

FIG. 11 shows the graphical representation of the GlycA/B signals in an NMR spectrum from blood serum, where an adjustment is made with characteristic frequency matrices for several proteins. The characteristic lines for different glycoproteins can be seen. The spectrum used can be recorded with any type of diffusion and relaxation pulse sequence that can use diffusion difference spectroscopy (DDS) for further differentiation of the diffusion regimes.

FIG. 12 shows an example of the adjustment according to the invention with a characteristic frequency matrix for the spectral range of the glycan groups (3.5-5.5 ppm) using the example of serotransferrin and immunoglobulin G (IgG). 12 (a) shows processed ¹H-NMR spectra of a serum sample spiked with serotransferrin (top) and isolated serotransferrin (bottom). 12 (b) shows processed ¹H-NMR spectra of a serum sample spiked with IgG (top) and isolated IgG (bottom).

The following shows examples of the analysis of samples from healthy controls compared to samples from patients with cardiogenic shock and COVID-19 compared to conventionally determined values.

FIG. 13 (a) shows the edited ¹H-NMR spectrum of blood serum from a COVID-19 patient. 13 (b) shows a GlycA/B section from the fitted NMR spectrum of blood serum and (c) the concentrations derived from this fitting procedure (wherein “measured by NMR”) compared to conventionally determined values (wherein “conventionally determined”), illustrating the quality of glycoprotein determination by the above-mentioned diffusion difference spectroscopy.

FIG. 13 shows how the spectra were deconvoluted using a series of characteristic frequencies to derive the concentrations of various glycoprotein biomarkers. FIG. 13 also shows that the relative concentrations of these proteins correlate well with those determined using conventional methods known to those skilled in the art, such as electrophoresis gel.

FIG. 14 shows examples of blood serum analysis for healthy controls, patients with cardiogenic shock, and COVID-19 patients. The contributions of individual glycoproteins as biomarkers, determined according to the invention, are shown.

FIG. 14 (a) shows haptoglobin, α-1-antitrypsin, ceruloplasmin, and complement factor C3 from the NMR analysis. 14 (b) shows the correlation of the analysis shown in FIG. 14 (a) with other parameters obtained from an NMR analysis of blood.

A statistical analysis of the spectra is shown in FIG. 14. FIG. 14 (a) shows the correlation of the glycoprotein contents determined using the method according to the invention with conventionally determined values. FIG. 14(a) also shows how the values for four glycoproteins are grouped into three groups for blood samples from healthy controls, patients with cardiogenic shock, and COVID-19 patients, demonstrating the value of such parameters as medical biomarkers.

FIG. 14 (b) shows correlations of such glycoprotein biomarkers with a panel of metabolites and lipoprotein biomarkers for COVID-19 compared to healthy controls. The diameter of the dots indicates varying degrees of correlation between the determined parameters. Strong correlations are found for various glycolysed acute phase proteins with LDL fractions (SPECIFIC EXAMPLES), showing that glycoprotein biomarkers correlate strongly with lipoprotein biomarkers.

FIG. 15 shows NMR spectra and assignments of glycan signals.

FIG. 15 a) shows the NMR spectrum of a blood sample recorded using a frequency-selective TOCSY pulse sequence (such as sTOCSY or sPerTOCSY, as shown in FIG. 18) to select the glycan signals, wherein the area outlined by a dashed box marks the excited frequency range around 3.7 ppm. The transfer of magnetization to other glycan protons is performed by a pulse sequence known to those skilled in the art as TOCSY, in which a sequence known as MLEV was used. The assigned glycan signals are marked in the spectrum, wherein the assignments are shown in the box on the right using symbolic representations of the glycan residues. The spectrum shows the anomeric signals of GlcNAc and Gal, the signals of Neu5Ac, and the fucose signal Q-CH₃ (Fuc).

FIG. 15 b) shows the NMR spectrum of a blood sample recorded with a frequency-selective TOCSY pulse sequence to select the glycan signals, wherein the area outlined by a dashed box marks the excited frequency range around 4.3 ppm. The transfer of magnetization to other glycan protons is achieved by a pulse sequence known as TOCSY. The assigned glycan signals are marked in the spectrum, wherein the assignments are shown in the box on the right using symbolic representations of the glycan residues. The spectrum shows additional fucose signals with different chemical shifts depending on the respective protein environment, enabling differentiation between different proteins.

FIG. 15 c) shows the NMR spectrum of a blood sample recorded using a frequency-selective TOCSY pulse sequence (not necessarily restrictive) to select the glycan signals, wherein the area outlined by a dashed box marks the excited frequency range around 4.85 ppm. The transfer of magnetization to other glycan protons is achieved by a pulse sequence known as TOCSY. The assigned glycan signals are marked in the spectrum, wherein the assignments are shown in the box on the right using symbolic representations of the glycan residues. The spectrum shows the Lewis antigen θ-CH₃-Fucose signal typical of cancer, wherein a distinction can be made between (Sialyl-)Lewis-Y/B and (Sialyl-)Lewis-X/A.

FIG. 16 shows the use of glycan biomarkers for the diagnosis of IBD (inflammatory bowel disease) based on a principal component analysis (PCA, PC=Principle Component).

FIG. 16 (a) shows the PCA based on the adjusted signal intensities of the corresponding glycan frequency matrix. The PCA was based on serum samples from 79 patients with acute IBD (Crohn's disease or ulcerative colitis) and 50 healthy controls (selected according to age, gender, and BMI). The scores plot of the principal component analysis shows a clear separation of the two groups and underscores the biomarker quality of the glycan signature.

FIG. 16 (b) shows five box plots for individual intensities of glycan signals resulting from computer-assisted fitting for the entire NAc signal (NAc), for the H8 and H9 protons of Neu5Ac, for the K and M H1 protons of Gal, for the M protons of Gal and O/P-H6 protons of GlcNAc, and for the Fuc Q-CH3 protons. The nomenclature is as shown in the box in FIG. 15. The box plots marked with 4 stars indicate q-values of <0.0001 in Mann-Whitney U tests followed by the false discovery rate procedure according to Benjamini, Krieger, and Yekutieli (Q=0.05).

FIG. 17 shows the use of glycan biomarkers for the diagnosis of Parkinson's disease (=Parkinson's syndrome, PS) based on a PLSDA analysis.

FIG. 17 (a) shows the PLSDA (partial least squares regression discriminant analysis) based on the adjusted signal intensities of the corresponding glycan frequency matrix. The PLSDA was based on serum samples from healthy controls, healthy subjects with heterozygous Parkin or PINK1 mutations ( ), idiopathic PS, and patients with homozygous or heterozygous Parkin/PINK1 PS. There is a clear separation between healthy controls and mutation carriers, whether diseased or not. In addition, there is a less pronounced separation between idiopathic PS and controls.

FIG. 17 (b) shows box plots based on the characteristic glycan signals that allow PS samples to be differentiated. The nomenclature is as shown in the box in FIG. 15. The stars indicate Kruskal-Wallis tests with Dunn's post-hoc tests followed by the false discovery rate procedure according to Benjamini, Krieger, and Yekutieli (Q=0.05), wherein * stands for a q-value of <0.05, ** for a q-value of <0.01, *** for a q-value of <0.001, and **** for a q-value of <0.0001.

FIG. 18 shows the use of glycan biomarkers for the diagnosis of HCC (hepatocellular carcinoma, HCC) based on a principal component analysis (PCA, PC=principle component) based on the adjusted signal intensities of the corresponding glycan frequency matrix. The PCA was based on serum samples from 80 patients with HCC and 20 healthy controls (selected according to age, gender, and BMI). The scores of the principal component analysis show a clear separation of the two groups and underline the biomarker quality of the glycan signature.

FIG. 19 (a) shows the box plots for samples from HCC patients corresponding to FIG. 18. Box plots are shown for 18 different glycan signals or ratios of glycan signals, which form the basis of the PCA in FIG. 18.

The box plots marked with stars indicate q-values in Mann-Whitney U tests followed by the false discovery rate procedure according to Benjamini, Krieger, and Yekutieli (Q=0.05), wherein * stands for a q-value of <0.05, ** for a q-value of <0.01, *** indicates a q-value of <0.001, and **** indicates a q-value of <0.0001.

FIG. 19 (b) shows the abbreviations for glycan signals and derived changes in the glycosylation profile in HCC patients. An upward-pointing arrow indicates increased levels of alpha-2,6-sialylated antennae, incompletely sialylated or galactosylated antennae, and Lewis antigen. A downward arrow indicates decreased levels of alpha-2,3-sialylated antennae. “Antennae” refers to the glycan structures shown. In various proteins, Fuc-Q is shown to be upregulated or downregulated.

FIG. 19 (c) Shows a Vulcano Plot Representation of the Glycan Biomarkers for Hcc.

FIG. 19 (d) shows the differences between various glycosylation signals of HCC in the ratio to healthy controls in units of standard deviations. The filled circles indicate statistical significance as described above.

FIG. 20 shows the use of glycan biomarkers for the diagnosis of MASLD (metabolic dysfunction-associated steatotic liver disease) based on a principal component analysis (PCA) based on the adjusted signal intensities of the corresponding glycan frequency matrix. The PCA was based on serum samples from 96 MASLD patients and 40 healthy controls (selected according to matching age, gender, and BMI). The scores of the principal component analysis show a clear separation of the two groups and underline the biomarker quality of the glycan signature.

FIG. 21 (a) shows the PLSDA (partial least squares regression discriminant analysis) based on the adjusted signal intensities of the corresponding glycan frequency matrix. The PLSDA was based on serum samples from healthy controls, MASLD patients with wild-type PNPLA3, and MASLD patients with a homozygous or heterozygous PNPLA3 mutation. The figure shows a clear separation of controls and homozygous mutation carriers, as well as a clear separation of these two groups from wild-type PNPLA3 and heterozygous MASLD patients.

FIG. 21 (b) shows the box plots for samples from MASLD patients corresponding to FIG. 21 (a). Box plots for four different glycan signals underlying the PLSDA in FIG. 21 (a) are shown. The stars indicate Kruskal-Wallis tests with Dunn's post-hoc tests followed by the false discovery rate procedure according to Benjamini, Krieger, and Yekutieli (Q=0.05), wherein * stands for a q-value of <0.05, ** for a q-value of <0.01, *** for a q-value of <0.001, and **** for a q-value of <0.0001.

FIG. 21 (c) shows characteristic differences between antennas of healthy and MASLD patients, wherein an increase in triantennary structures and alpha-2,3-sialylated antennas is observed.

The method according to the invention goes beyond previous analyses of glycoproteins in that it allows the contributions of individual glycoproteins or protein classes (e.g., in the case of IgG) or glycosylation patterns to be shown in detail. This is achieved by determining a characteristic frequency matrix for each glycoprotein, which is then fitted to the spectra of blood serum or plasma. The invention has recognized that the glycan region of NMR spectra should also be used as a further feature of the glycoprotein type and glycan structure.

According to the invention, ¹H-NMR-NMR spectra of blood serum or plasma are used to derive biomarkers for risk assessment or clinical diagnosis of diseases. Specific proteins or classes of proteins that are very common in blood and are referred to as acute phase proteins are identified and/or quantified using a matrix of chemical shifts characteristic of each protein. The biomarkers are derived by electronic unfolding of characteristic protein signals using a matrix of characteristic chemical shifts for individual signal components. The signal components represent individual acute phase proteins or classes of glycosylated acute phase proteins. The measurements of acute phase proteins can be used as clinical biomarkers for clinical disease states.

The analysis can be used for medical diagnostic purposes, as demonstrated in exemplary cases with samples from healthy controls and patients with cardiogenic shock or COVID-19. The more detailed glycoprotein parameters can support medical diagnosis or differentiation between patients.

The following description of the procedure for carrying out the method according to the invention is not intended to limit the generality of the teaching.

Serum or plasma was prepared for blood sample analysis using methods and protocols established in the clinic or laboratories; for serum, this involved centrifugation after a period of blood coagulation, and for plasma (EDTA or heparin plasma), only centrifugation of the samples.

The frozen serum or plasma samples were thawed and then mixed 1:1 with a 75 mM phosphate buffer, then shaken for one minute. 600 μL of sample was then transferred to a 5 mm NMR tube. The NMR analysis was then measured using a 600 MHz NMR spectrometer (Bruker Avance III HD spectrometer with a room temperature sample head optimized for ¹H. The spectrometer was calibrated for these investigations as described in Dona et al [Dona, A. C., Jiménez, B., Schäfer, H., Humpfer, E., Spraul, M., Lewis, M. R., et al. (2014). Precision High-Throughput Proton NMR Spectroscopy of Human Urine, Serum, and Plasma for Large-Scale Metabolic Phenotyping. Anal. Chem. 86, 9887-9894. doi:10.1021/ac5025039].

### LIST OF FIGURES

FIG. 1: Schematic representation of the assignment of glycan, GlycA, and GlycB signal groups in an edited ¹H-NMR spectrum of blood serum

FIG. 2: Structure of a glycan unit consisting of several sugar units

FIG. 3: Structures of the individual glycan units that contribute to the NMR signals of GlycA and B

FIG. 4: Shell signal of GlycA and GlycB with marked contributions from Neu5Ac, GlcNAc, and methionine to illustrate the assignment scheme from FIG. 1

FIG. 5: NMR spectra illustrating the accuracy of the assignments underlying the use of GlycA and GlycB for the analysis of glycan components in glycoproteins

FIG. 6: Contributions of common N-glycans, acute phase proteins in human blood

FIG. 7: Contributions of low-abundance N-glycans to the GlycA/B signal

FIG. 8: Typical pulse sequences in NMR spectra in glycoprotein analysis

FIG. 9: Typical spectral unfolding using line shape fitting, using the GlycA/B spectral range as an example

FIG. 10: Illustration of a frequency matrix for the GlycA/B region using the example of serotransferrin

FIG. 11: GlycA/B region in an NMR spectrum adjusted with characteristic frequency matrices according to the invention

FIG. 12: Illustration of two frequency matrices for the glycan region using the example of serotransferrin and IgGs

FIG. 13: Analysis according to the invention of samples from healthy controls and samples from patients with cardiogenic shock and COVID-19 compared to a classical approach

FIG. 14: Analysis of blood samples from healthy controls and samples from patients with cardiogenic shock and COVID-19

FIG. 15: NMR spectra and assignments of glycan signals

FIG. 16: Use of glycan biomarkers for the diagnosis of IBD based on principal component analysis

FIG. 17: Use of glycan biomarkers for the diagnosis of Parkinson's disease based on PLSDA analysis

FIG. 18: Use of glycan biomarkers for the diagnosis of HCC based on a principal component analysis based on the adjusted signal intensities of the corresponding glycan frequency matrix

FIG. 19: (a): Box plots for samples from HCC patients corresponding to FIG. 18
(b): Abbreviations for glycan signals and derived changes in the glycosylation profile in HCC patients
(c): a Vulcano plot representation of glycan biomarkers for HCC
(d): the differences between various glycosylation signals of HCC in relation to healthy controls in units of standard deviations

FIG. 20: Use of glycan biomarkers for the diagnosis of MASLD based on a principal component analysis based on the adjusted signal intensities of the corresponding glycan frequency matrix

FIG. 21: (a): PLSDA based on the adjusted signal intensities of the corresponding glycan frequency matrix
(b): Box plots for samples from MASLD patients corresponding to (a)

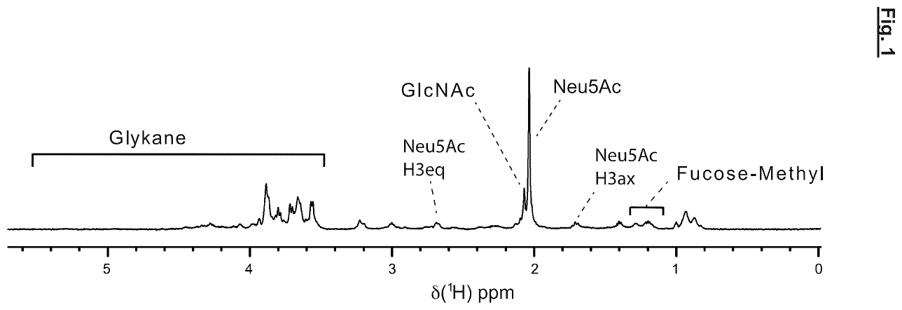

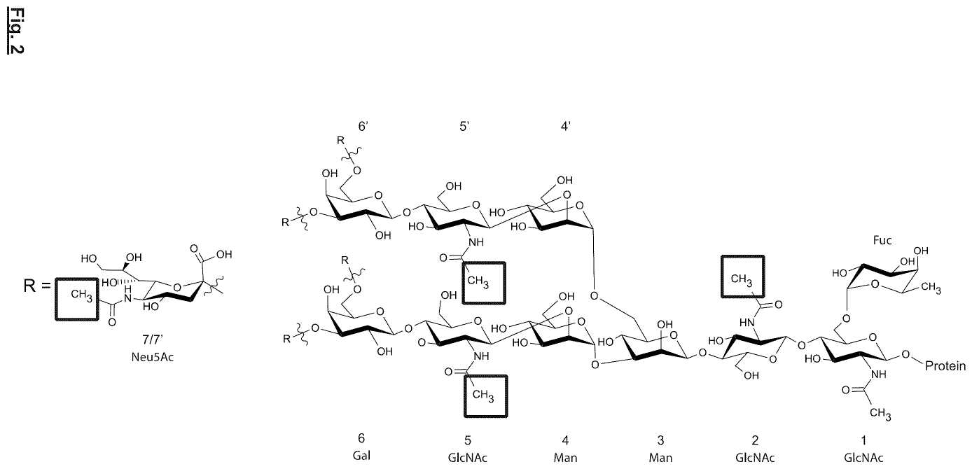

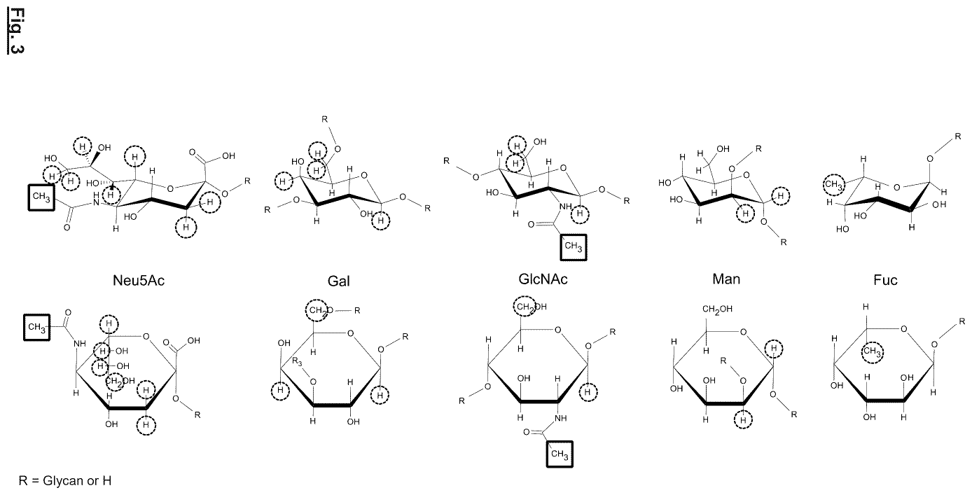

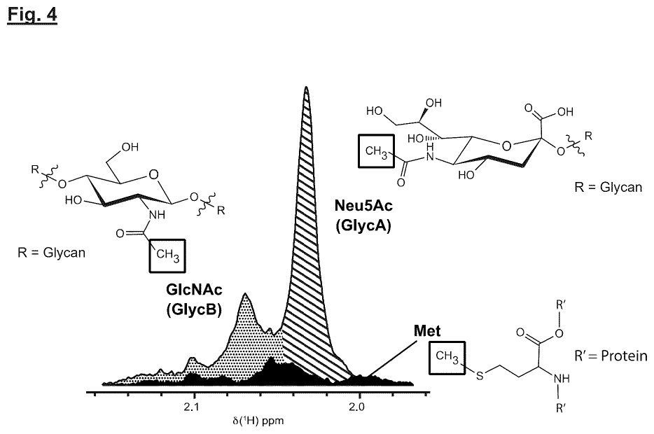

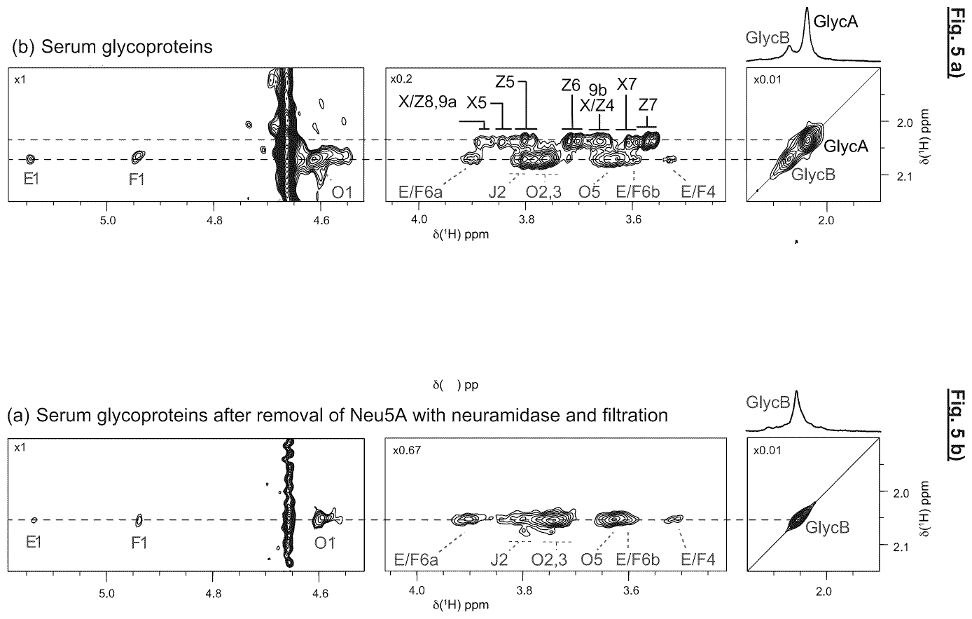

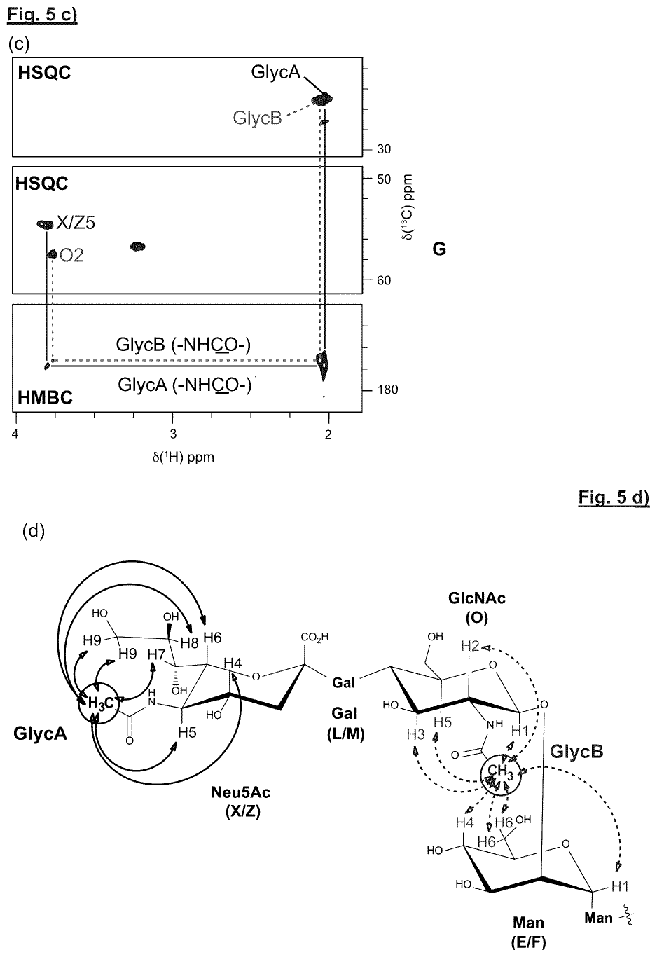

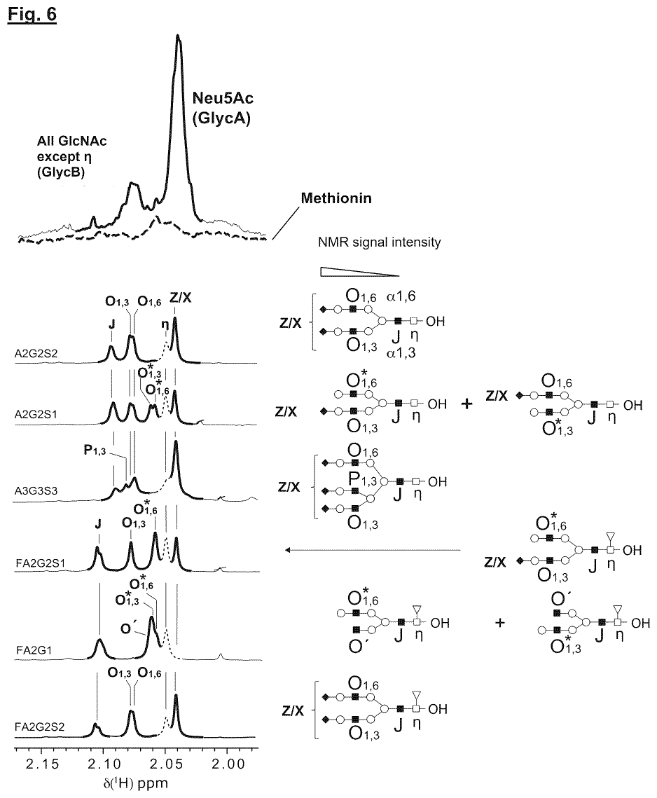

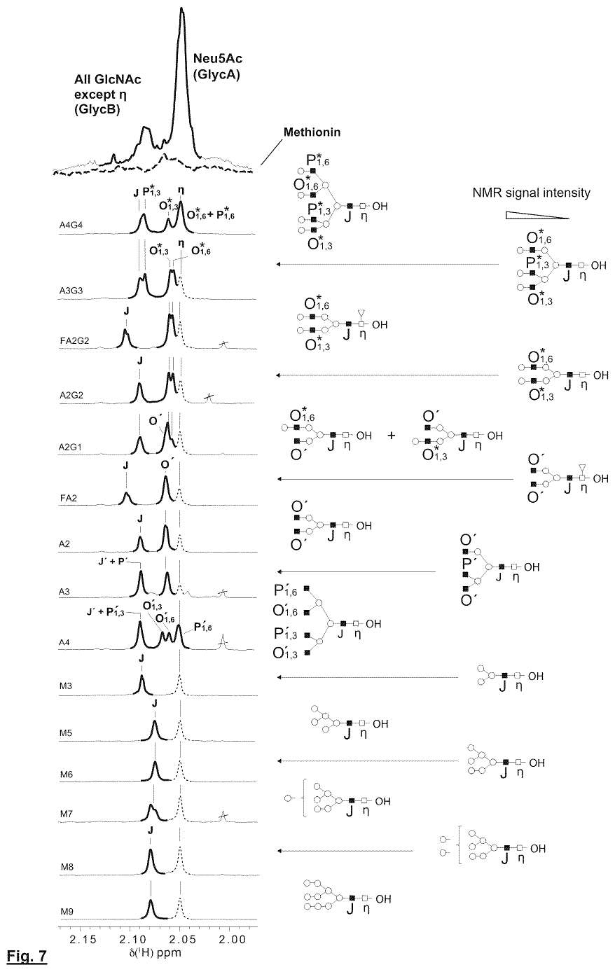

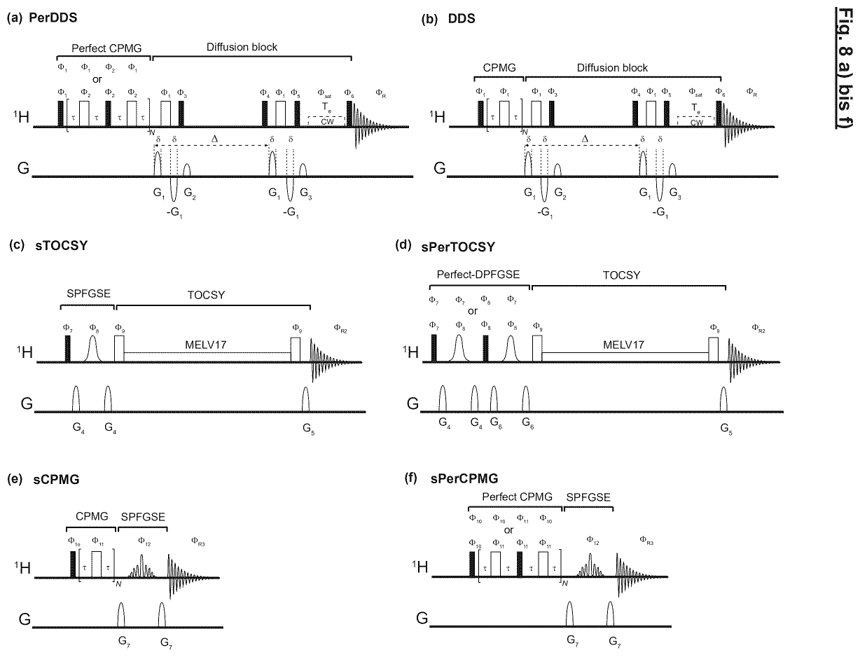

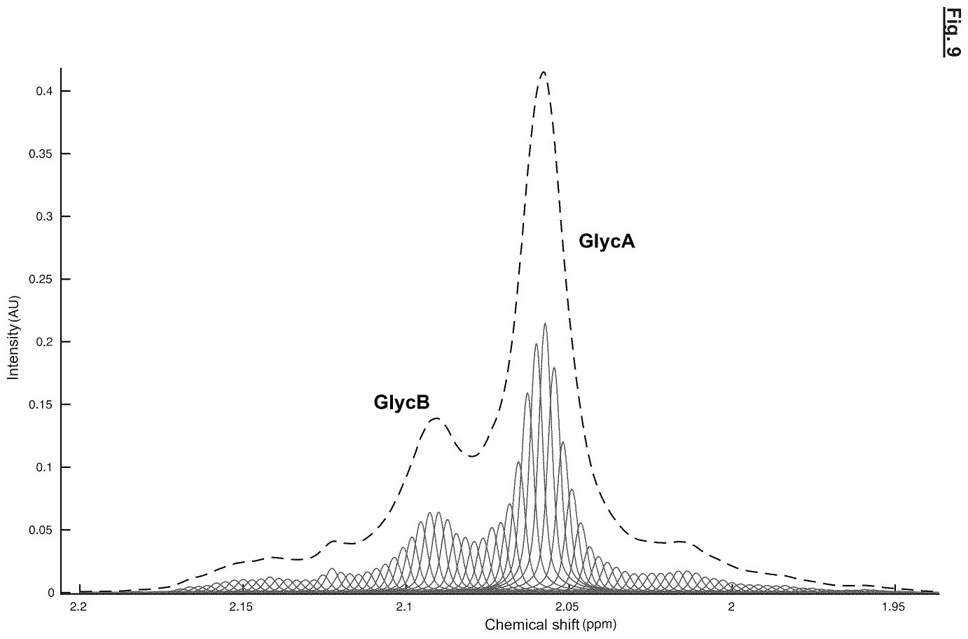

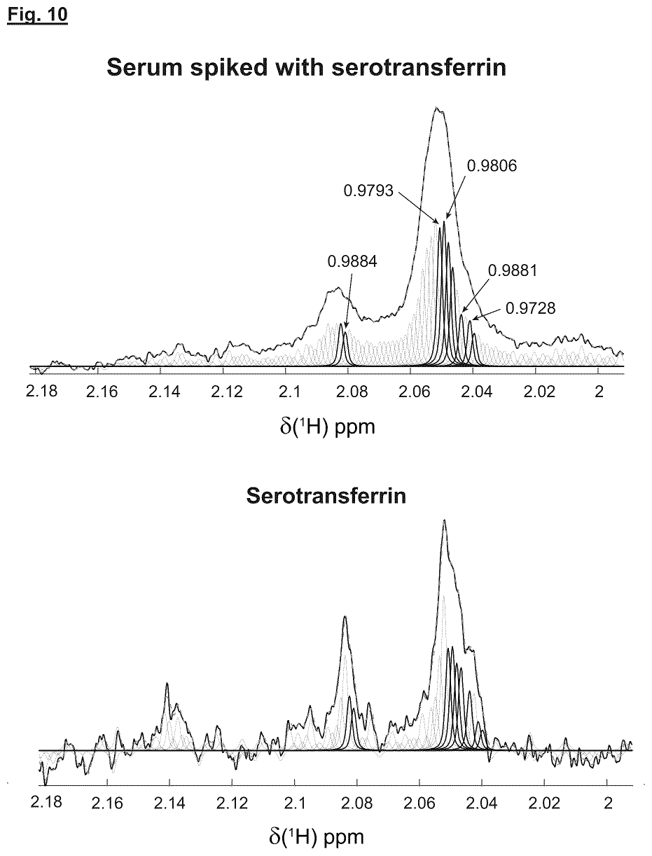

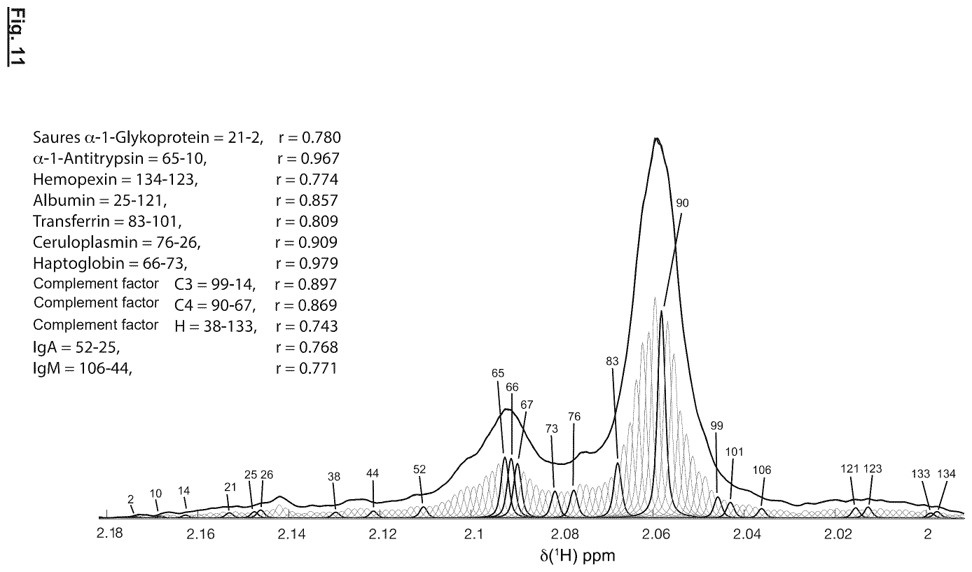

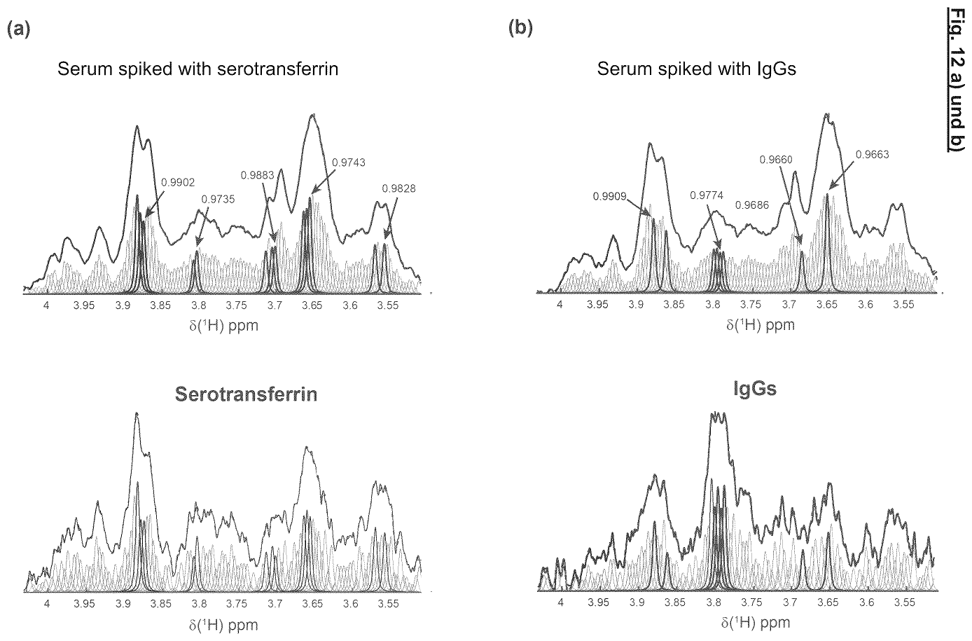

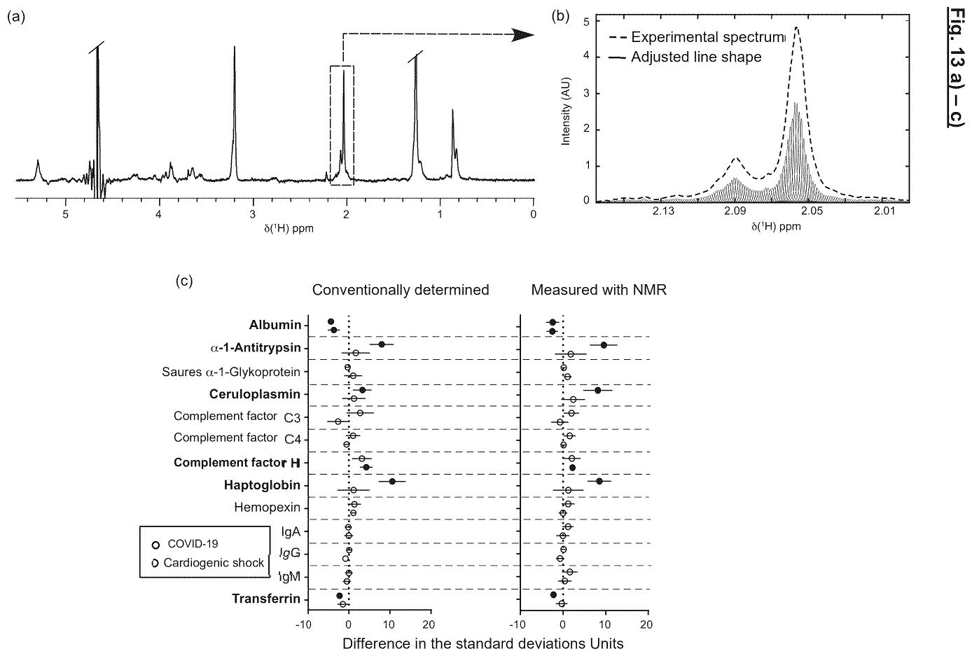

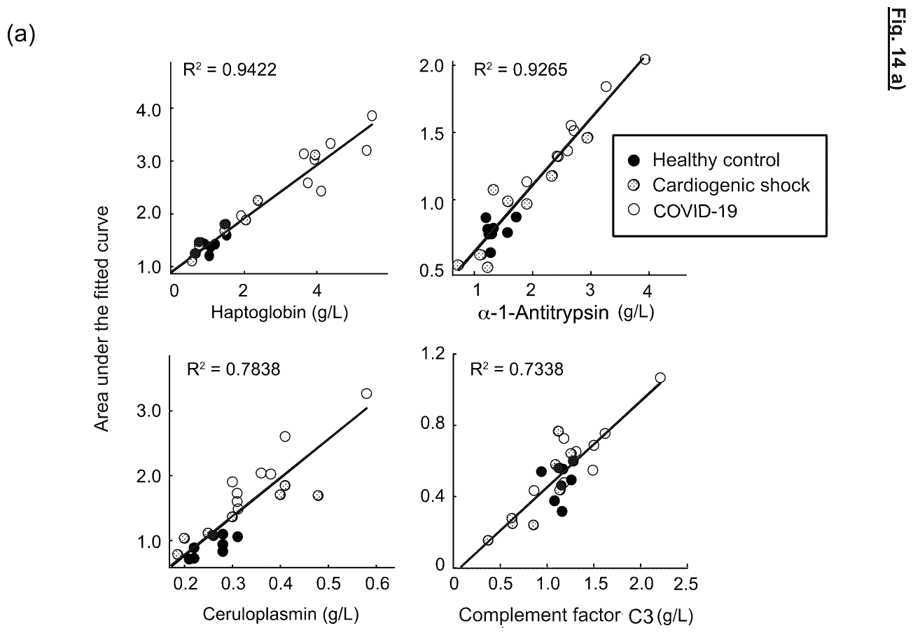

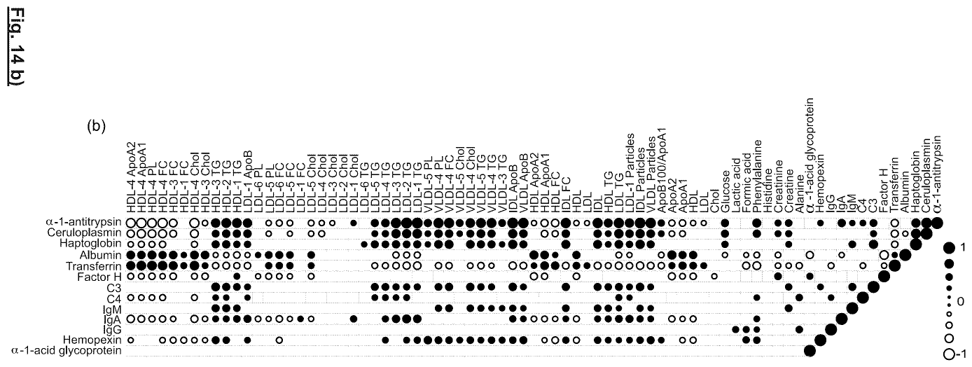

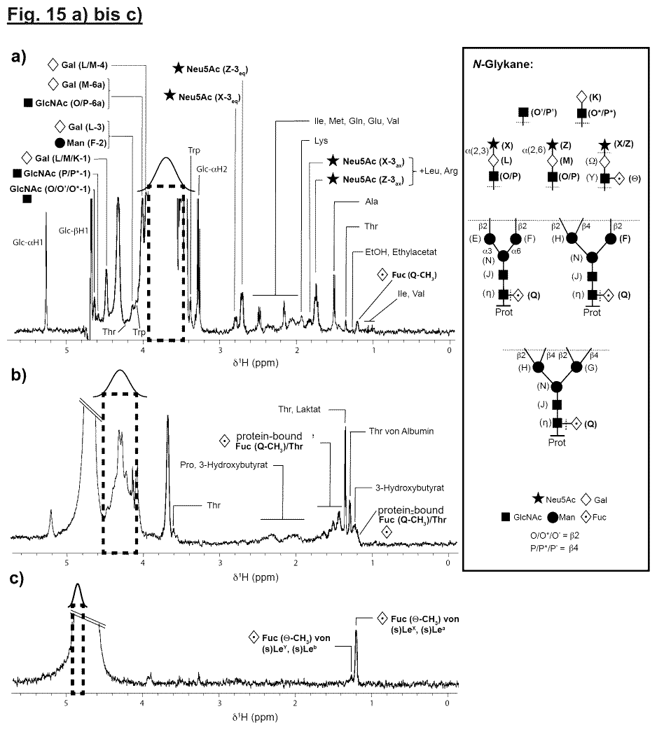

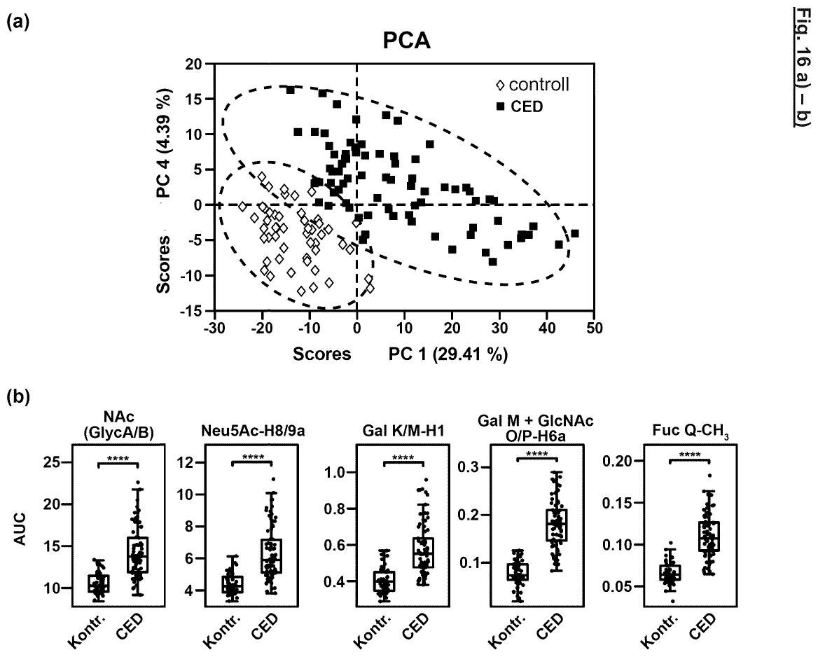

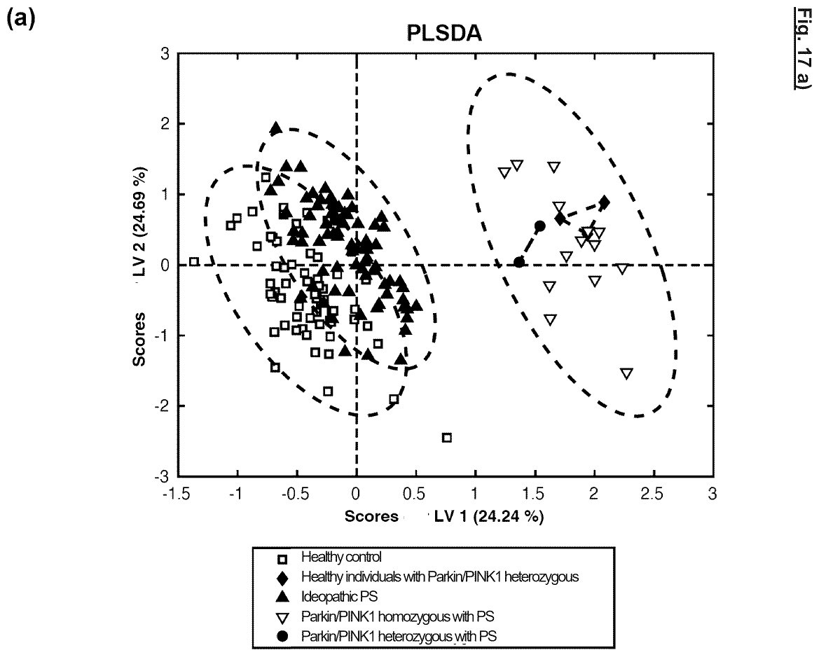

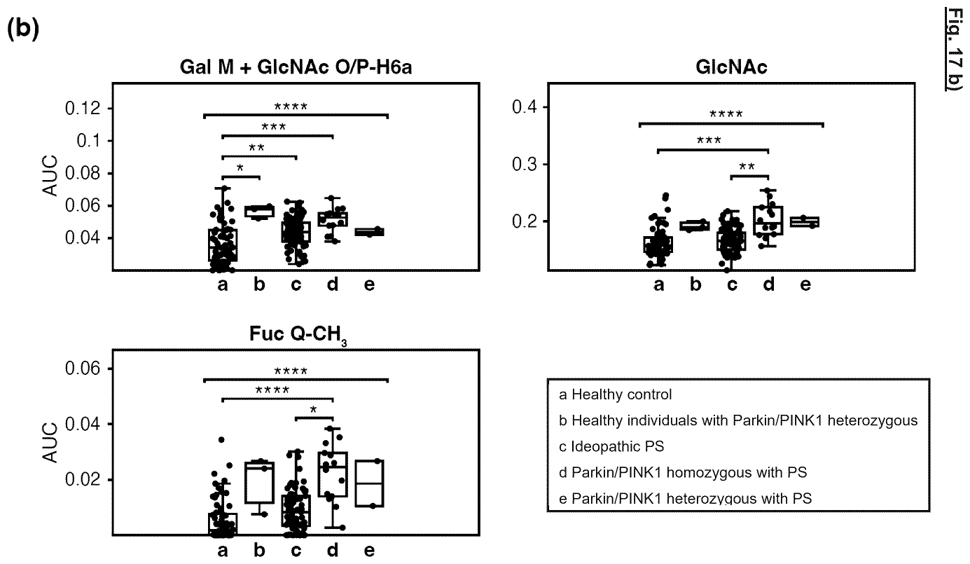

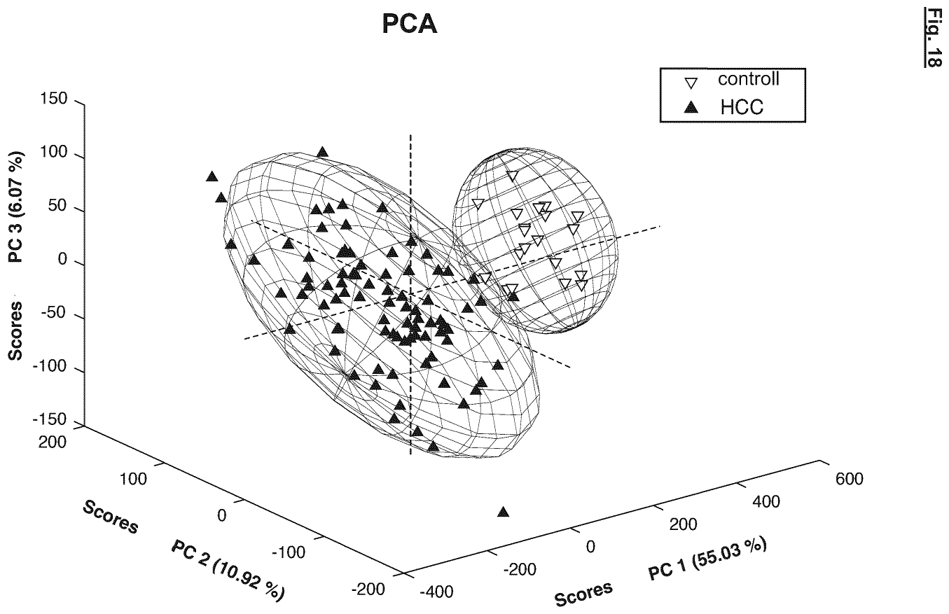

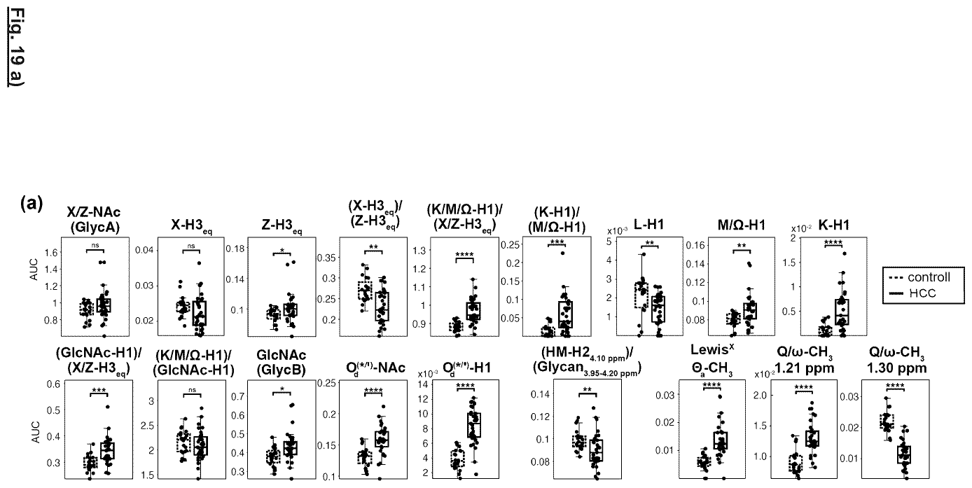

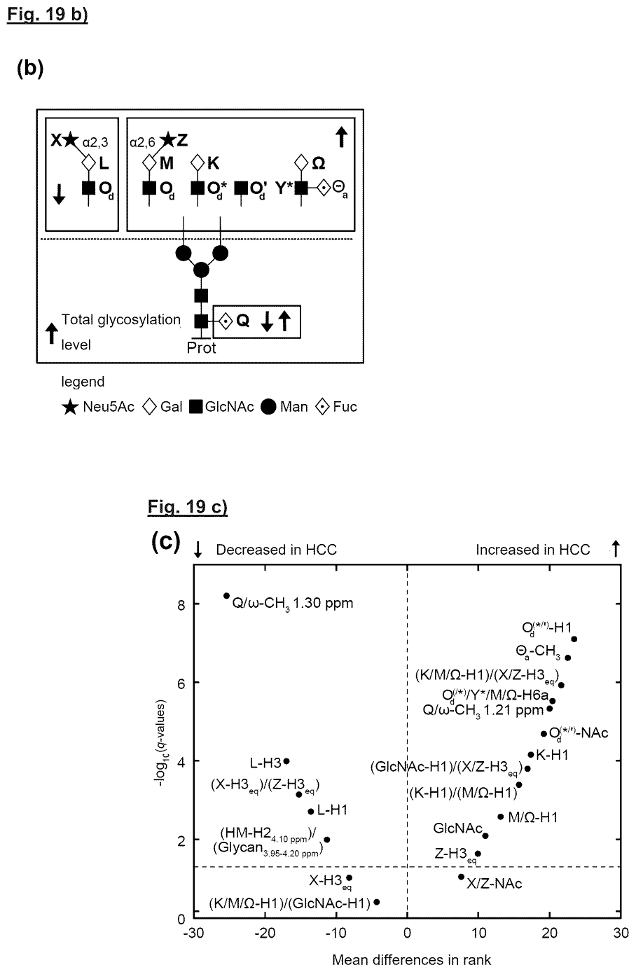

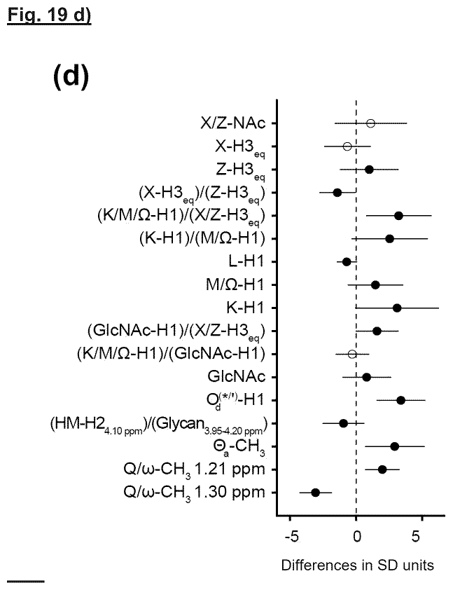

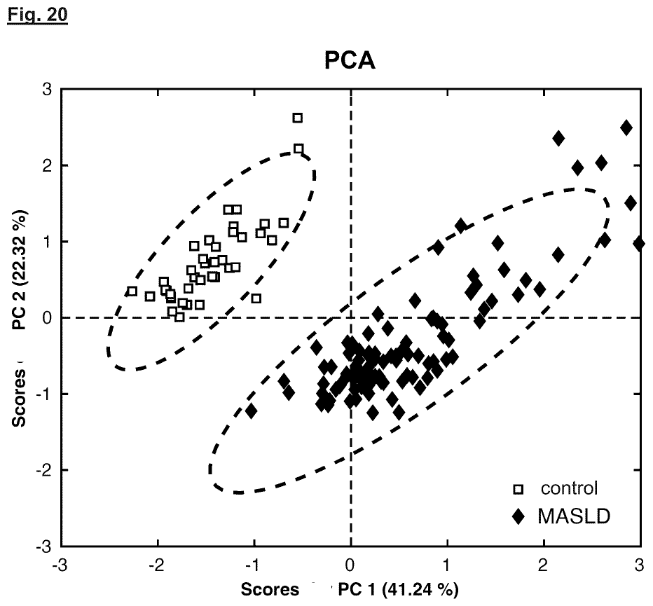

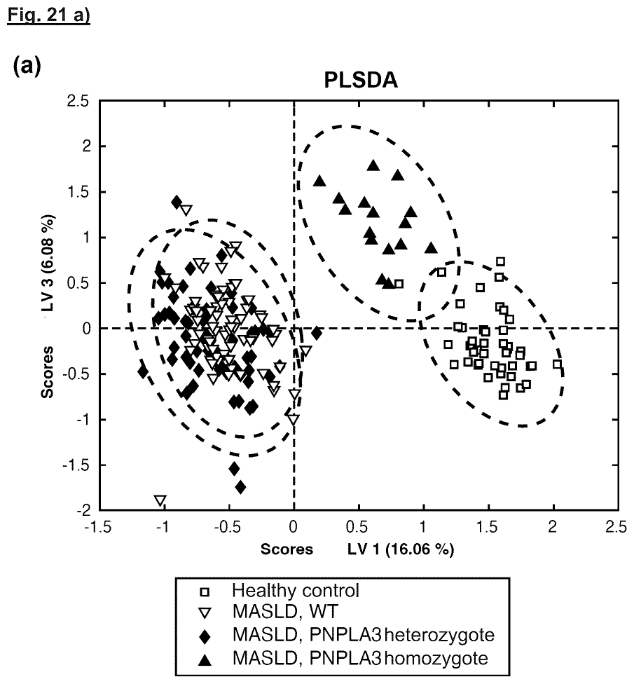

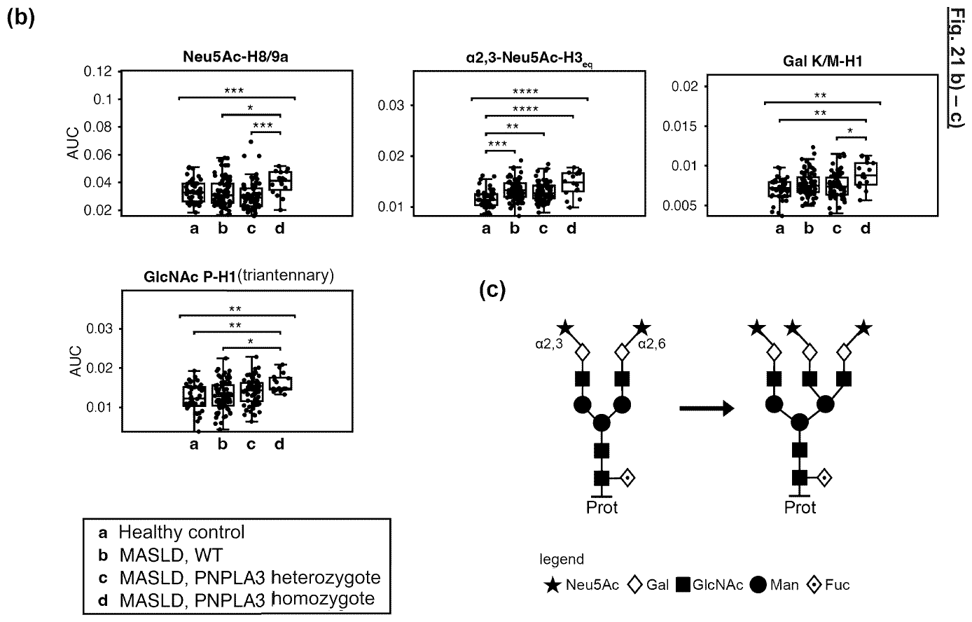

1. A method for measuring the contributions of at least one of individual glycoproteins and glycosylation patterns as biomarkers, derived from an analysis of NMR spectra of at least one of blood serum and blood plasma, comprising the steps:
i. selecting glycoproteins from a sample of at least one of blood serum and blood plasma based on at least one of their specific diffusion, relaxation properties and frequencies and/or J-couplings;
ii. determining the glycan, GlycA, and GlycB signal groups in an NMR spectrum, wherein the GlycA signal group is formed by methyl groups of Neu5Ac and wherein the GlycB signal group is formed by methyl groups of GlcNAc units and wherein the glycan signal group is between 3.5 and 5.5 ppm and the Neu5Ac-H3eq signal group between 2.6 and 2.9 ppm, and the Neu5Ac-H3ax signal group is between 1.6 and 1.88 ppm, the GlycA signal group is between 2.0 and 2.07 ppm, and the GlycB signal group is between 2.07 and 2.2 ppm, and wherein the signals are assigned on the basis of the diagram shown in FIG. 1;
iii. adapting characteristic frequency matrices for individual glycoproteins or glycoproteins previously grouped into classes to the determined signal groups using mathematical methods; and
iv. determining the contributions of individual glycoprotein biomarkers by deconvolution of the spectrum using the frequency matrices adapted in step iii and addition of a previously assigned number of mathematical functions.

2. The method according to claim 1, wherein the selection of glycoproteins from a blood serum sample based on at least one of their specific diffusion and relaxation properties (step i) is performed using diffusion difference spectroscopy (DDS).

3. The method according to claim 1 further comprising multiplication by a scaling factor determined by a reference measurement is to indicate concentrations in mg/ml, wherein the multiplication may be performed before the deconvolution of the spectra or after step iv.

4. A computer program product for evaluating biological in vitro blood plasma or serum samples, comprising a non-volatile computer-readable storage medium with computer-readable program code embodied in the storage medium, wherein the computer-readable program code includes:
computer-readable program code that unfolds a composite NMR spectrum of a fitting region of a blood plasma or serum sample from an individual, wherein the computer-readable program code uses a characteristic frequency matrix, which includes unfolding models for different proteins, to decompose the composite NMR spectrum into signals with components of (a) GlycA, (b) GlycB, (c) glycan protons, and (d) methyl group protons, and wherein the program code defines the frequency ranges for determining the glycan, GlycA, and GlycB signal groups, wherein the glycan signal group is between 3.5 and 5.5 ppm and the Neu5Ac-H3eq signal group is between 2.6 and 2.9 ppm and the Neu5Ac-H3ax signal group between 1.6 and 1.88 ppm, the GlycA signal group between 2.0 and 2.07 ppm, and the GlycB signal group between 2.07 and 2.2 ppm, and
a scheme for assigning the signals according to FIG. 1,
and wherein the program code adds a defined number of mathematical functions at frequencies known from the characteristic frequency matrix in order to determine the percentages of the individual proteins or protein groups.

5. (canceled)

6. A method for diagnosis of a disease, comprising measuring the contributions of at least one of individual glycoproteins and glycosylation patterns as biomarkers according to claim 1.

7. The method according to claim 6, wherein the disease is cardiogenic shock, COVID-19, obesity, diabetes, metabolic syndrome, heart failure, rheumatoid arthritis, chronic obstructive pulmonary disease (COPD), systemic lupus erythematosus, psoriasis, cystic fibrosis, polycystic ovary syndrome, Cushing's disease, respiratory tract infection, HIV, primary aldosteronism, inflammatory bowel disease, chronic kidney disease, Kawasaki disease, cognitive decline, or cancer.

8. A method for diagnosis of a disease, comprising subjecting biological in vitro blood plasma or serum samples to computer analysis using a computer program product according to claim 4.

9. The method according to claim 8, wherein the disease is cardiogenic shock, COVID-19, obesity, diabetes, metabolic syndrome, heart failure, rheumatoid arthritis, chronic obstructive pulmonary disease (COPD), systemic lupus erythematosus, psoriasis, cystic fibrosis, polycystic ovary syndrome, Cushing's disease, respiratory tract infection, HIV, primary aldosteronism, inflammatory bowel disease, chronic kidney disease, Kawasaki disease, cognitive decline, or cancer.
